(19) United States
(12) Patent Application Publication
(10) Pub. No.: US 2010/0028871 A1
(43) Pub. Date: Feb. 4, 2010

(54) Light-inducible and rhythmically expressed genes and method for screening substances that affect an internal clock system

(75) Inventor: Masumi Abe, Chiba (JP)

Correspondence Address: MORRIS MANNING MARTIN LLP, 3343 PEACHTREE ROAD, NE, 1600 ATLANTA FINANCIAL CENTER, ATLANTA, GA, 30326, US

(73) Assignee: National Institute of Radiological Sciences, Chiba-shi, Chiba (JP)

(21) Appl. No.: 12/227,522

(22) PCT Filed: May 29, 2007
(86) PCT No.: PCT/JP2007/061257

§ 371 Date: Feb. 25, 2009

Related U.S. Application Data
(60) Provisional application No. 60/809,364, filed on May 31, 2006.

(51) Int. Cl. C12Q 1/68

(52) U.S. Cl. 435/6

(57) ABSTRACT

A method for screening a substance that affect an internal clock system in an organism, comprising the detection of the expression level of a gene exhibiting a light-inducible and rhythmic expression in a circadian manner, which is typically expressed in the suprachiasmatic nucleus of mammalian, such as for example, Dusp4, Snk, Slc39a6 or Nnat.

### FIELD

This invention relates to light-inducible and rhythmically expressed genes and method for screening substances that affect an internal clock system.

### BACKGROUND

Most biological phenomena, including behavior and metabolic pathways, are governed by an internal clock (biological clock) system that is circadian (i.e. with a period of approximately 24 hours), and is reset by light exposure from outside.

Conceptually, the biological clock is comprised of three pathways: the input, oscillator and output. The oscillator generates a signal with a period of about 24 hours which is reset by light stimuli via the input pathway, and which controls biological phenomena via the output pathway.

Attempts to identify rhythmically expressed genes have been conducted using micro-arrays and hundreds of cycling genes have been identified in the suprachiasmatic nucleus (SCN) and even in peripheral tissues, indicating that many kinds of cells, not just the SCN, have an internal biological clock system in mammals [10]. The precise functions of the rhythmically expressed genes are unclear. They are thought to be implicated in the oscillator and output systems.

We developed a new gene expression profiling procedure called high coverage expression profiling (HiCEP) [3]. HiCEP is an amplified fragment length polymorphism (AFLP)-based method that is sensitive enough to detect one transcript copy per cell and capable of detecting unknown transcripts as well as known genes [14]. It also features a false positive rate less than 4% [14]. Previous AFLP-based methods yielded many false positives, making them unsuitable for profiling analysis. The low false positive rate in HiCEP reduced the number of peaks and enabled us to assign peaks to specific genes unequivocally. We could therefore use four-nucleotide recognition enzymes, MspI and MseI, giving us an estimated coverage of 85.6%. Additionally, only a small amount of starting material is required for analysis, and the method can distinguish a 1.2-fold difference in gene expression. A precise delineation of rarely and moderately expressed transcripts in which most of the transcription regulation factor-coding genes are included is possible with this procedure. This is another advantage of this procedure, since the hybridization-based procedures are not ideal for this purpose. Here we present an analysis of the mouse SCN just after light exposure using HiCEP.

Because several key genes in the oscillator such as mammalian Period (Per) 1 and Per2 are also light-inducible, indicating some overlapping between the input and oscillation mechanisms [1], we hoped to identify some of the genes in the oscillator itself [12].

### SUMMARY

Thus, the present invention is related to the following methods:

1. A method for screening a substance that affects an internal clock system in an organism, comprising the detection of the expression level of a gene exhibiting a light-inducible and rhythmic expression in a circadian manner.

2. A method for screening according to Claim 1, based on change in the expression level of the gene exhibiting a light-inducible and rhythmic expression in a circadian manner in the presence of the substance.

3. A method for screening according to Claim 1, the gene is expressed in the suprachiasmatic nucleus of mammalian.

4. A method of screening according to Claim 1, the gene is selected from the group consisting of Dusp4, Snk, Slc39a6, Nnat and any combination thereof.

5. A method of screening according to Claim 1, comprising the steps of:

(a) contacting a cultured cell line derived from the suprachiasmatic nucleus in a brain with a substance to be screened;

(b) detecting change of the expression level of the gene; and

(c) selecting the substance that affects the expression level of the gene.

6. A method of screening according to any one of Claims 1-5, wherein the expression level of a gene is detected by determining an amount of a transcript of the gene.

7. A method of screening according to Claim 6 wherein the amount of the transcript of the gene is determined by obtaining a gene expression profile by means of HiCEP procedure.

8. A method of screening according to Claim 6 wherein the amount of the transcript of the gene is determined by PCR.

9. A method according to Claim 7 or 8, wherein the transcript of the gene comprises the base sequence selected from the group consisting of:

(a) a base sequences represented by SEQ ID No. 1, 2, 3 and 4, respectively:

(b) a base sequence that hybridizes under strict conditions with a complementary base sequence of any one of the base sequence of (a); and

(c) a base sequence having homology of 80% or more with any one of the base sequence of (a).

10. A method of screening according to any one of Claims 1-5, wherein the expression level of a gene is detected by determining an amount of one or more proteins encoded by the gene or any part thereof.

11. A method for screening according to Claim 5, the protein is selected from the group consisting of:

(a) a protein encoded by the gene of Dusp4, Snk, Slc39a6 and Nnat, respectively;

(b) a protein having replacement or substitution, deletion or addition of one or a few amino acids in an amino acid sequence of any one of the protein of (a); and

(c) a protein consisting of an amino acid sequence having homology of 80% or more with any one of the amino acid sequence of the protein of (a).

### BRIEF DESCRIPTION OF THE DRAWINGS

FIG. 1. Flow of the processes of the identification of light-inducible genes from mouse SCN.

FIG. 2. Time-course of light induction of Slc39a6 and Dusp4 transcripts at CT15 detected by HiCEP and real-time PCR, and the light induction of them at various CTs observed by real-time PCR. Y-axis indicates the relative amount of RNA. X-axis indicates time, minutes, after the light exposure at CT15. The solid and closed circles indicates the mean values of RNA amount in the SCN, and the broken and open circles indicate those in the cortex (n=2). The light incubation at various CTs represents the analysis by real-time PCR of whether the induction occurs during subjective day or night. The filled and open bars indicate the relative RNA amount at 45 min after light exposure in the SCN, respectively.

FIG. 3. Time-course of light induction of Nnat and Snk transcripts at CT15 detected by HiCEP and real-time PCR, and the light induction of them at various CTs observed by real-time PCR. The details of the figure are the same as those of FIG. 2.

FIG. 4. Subjective-night specificity and localization (A-D). Analysis by real-time PCR of whether the induction occurs during subjective day or night in the SCN and the cortex. Four genes, C/EBP beta, Nr4a2, Btg2 and Snk are shown in A, B, C, and D, respectively. The filled and unfilled bars indicate the mean induction rate (Relative mRNA level) of mRNA at 45 min after light exposure in the SCN and the cortex, respectively. Standard deviations are shown (n=2). The Y-axis indicates induction rate when the fluorescent intensity at each CT in each tissue without light exposure is defined as 1.0.

(E-H) Localization and subjective night-specific induction detected by in situ hybridization. Representative autoradiograms of in situ hybridization of the brain containing the SCN and the results of quantitative analysis with C/EBP beta (D), Nr4a2 (E), Btg2 (F) and Snk probes are shown. The Y-axis indicates induction rate when the intensity at each CT without light exposure is defined as 1.0. Data values are the means±standard errors of three animals. We confirmed that sense probes for each gene did not give any signals (data not shown).

FIG. 5. Analysis of rhythmic expression of light-inducible transcripts by real-time PCR. Data for Per1 (A), Fosl2 (B), Dusp4 (C), Snk (D), Slc39a6 (E) and Nnat (F) are shown. The maximal value is expressed as 100%. Data values are the means±standard errors of three RNA pools from independent mice.

### DETAILED DESCRIPTION

In order to understand the molecular basis of the resetting mechanism of the clock, we attempted to isolate light-inducible transcripts in the SCN, where the master clock resides, using a new gene expression profiling procedure. We identified 87 such transcripts, successfully cloned 60 of them and confirmed their light inducibility. Six of the 60 were already known to be light inducible and 17 are protein-coding transcripts registered in the public database that were not known to be light inducible. Induction is subjective night-specific in most of the transcripts. Interestingly, 6 of the transcripts exhibit rhythmic expression in a circadian manner in the suprachiasmatic nucleus.

The present invention was made based on the above findings.

A substance that is screened by the above methods may affect an internal clock system in an organism such as mammalian including mouse and human in any way or manner, causing or inducing any change in the system, such as, for example, controlling, regulating, deregulating, accelerating, and delaying the system.

The present screening methods are therefore preferably and advantageously based on change such as increase or decrease in the expression level of one or more genes exhibiting a light-inducible and rhythmic expression in a circadian manner, or in its expression profile, by comparing those in the presence and the absence of the substance to be screened.

The gene exhibiting a light-inducible and rhythmic expression in a circadian manner is expressed, for example, in the suprachiasmatic nucleus in the brain of organism such as mammalian including mouse and human.

The representative examples of the above genes are selected from the group consisting of Dusp4, Snk, Slc39a6, Nnat and any combination thereof.

DUSP4 (Gene ID:319520 (NM 176933), also called MAP kinase phosphatase-2, is a phosphatase in the MAPK signal pathway, playing a role in inactivating active MAPK. The MAPK (ERK, JNK, p38) signal pathways play roles in the entrainment of circadian rhythm to environmental rhythm, and oscillation of MAPK activity has been suggested in the SCN [8]. Inactivation of the MAPK pathway after activation in response to light exposure may also induced by light exposure. Furthermore, the circadian character of Dusp4 may contribute to the rhythmic character of MAPK activity in the SCN.

Snk (GeneID:20620 (NM_{—}152804.1) was isolated as a kinase induced by serum shock [13]. This gene belongs to the polo kinase family, playing a role in cell cycle control, and four paralogues have been identified in mammals. Furthermore, functions have been suggested for the neuronal cells. D Pak and his colleagues identified SNK as a binding partner for Spine associated Rap GTP activating protein (SPAR) in the synapse [9]. Phosphorylation of SPAR molecules by SNK induces degradation of SPAR in the ubiquitin pathway, and the spine structure made of SPAR also degrades. The authors suggest that the interaction of SNK and SPAR causes the remodeling of the spine structure in the synapse.

Slc39a6 (GeneID:319520 (NM_{—}139143.1) belongs to the subfamily of zinc transporters [15], but its precise function is unclear. Rhythmic expression of some members has been reported [10].

Nnat (GeneID: 18111 (NM_{—}180960) is small molecule of less than 100 amino acids and five types of alternative transcripts for it have been identified [5]. It is an imprinting gene and expresses transiently uring neurogenesis, but its precise role is unclear. Interestingly, it resides in the anti-sense strand of an intron of the Bladder cancer associated protein (Blcap) gene [2]. Blcap also expresses rhythmically with the same phase as the Nnat transcripts in the SCN [10]. We noticed the rhythmic expression of Blcap transcripts during an in situ hybridization experiment by chance, because we detected SCN-specific rhythmical expression not only by anti-sense probes but also by sense probes. Approximately two hours' difference was observed between the expression phases of neuronatin and Blcap, suggesting gene-expression regulation at the transcription level.

The detection of the expression of the gene may be carried out in vivo, i.e., in a living body, or in vitro, i.e., in cultured cell lines.

The substance to be screened or tested according to the present methods may be accordingly administered to a test animal according to any known manner, or added or supplemented to a culture medium used in the culture of the cell lines.

For example, the method may comprise contacting a cultured cell line derived from the suprachiasmatic nucleus in the brain with a substance to be screened, detecting change of the expression level of the gene, and selecting the substance that affects the expression level of the gene or its expression profile.

The detection of the expression level of the gene exhibiting a light-inducible and rhythmic expression may be carried out in any stage of its expression such as transcription and translation.

The detection of the expression level of the gene may be carried out quantitatively, semi-quantitatively or qualitatively in accordance with a method or mechanism for the detection.

The detection of the expression level of the gene exhibiting a light-inducible and rhythmic expression in a circadian manner may be carried our by determining an amount of one or more transcripts (mRNA or cDNA) of the gene(s) by means of any method known for those skilled in the art, such as, for example, PCR methods such as real-time RT-PCR, in situ hybridization and a micro-array chip (DNA chip), or by obtaining a gene expression profile by means of HiCEP procedure. The screening or detection may be made at any predetermined time during a day, or at appropriate intervals during the time course.

The above transcript may correspond to a full length or any part of the gene, and therefore consist of the base pairs in any length.

Those skilled in the art may be easily and optionally design and prepare primers or probes that are used in the above methods by means of any known manner such as using a commercial software for primer designing in accordance with the information on the base sequences of the genes. They consists of base sequences with a number enough to specifically hybridize with their template, for example, 15-40 base pairs. Such primers or probes may be labeled with any substances known for those skilled in the art.

The specific steps of the HiCEP procedure are described in detail in, for example, WO02/48352 and WO2005/118791.

Especially, the number of cells that are obtained from pathological samples, microtissues, microanimals, etc. for the HiCEP may be significantly reduced by carrying out the following methods.

A method for the preparation of gene expression profile comprising:

(a) a step of synthesizing a single-stranded cDNA whose 5′ end is fixed to solid phase or which has a tag substance added to its 5′ end with poly(A) RNA as a template;

(b) a step of synthesizing a double-stranded cDNA with the single-stranded cDNA synthesized in the step (a) as a template;

(c) a step of cleaving the double-stranded cDNA prepared in the step (b) with a first restriction enzyme X;

(d) a step of purifying a 3′ end fragment that is fixed to the solid phase or has the tag substance added thereto from the double-stranded cDNA fragments prepared in the step (c);

(e) a step of preparing a double-stranded cDNA fragment having an X promoter-adaptor bound to its 5′ end by binding the X promoter-adaptor to a cleavage site with the first restriction enzyme X in the fragment purified in the step (d), wherein the X promoter-adaptor comprises a sequence complementary to the cleavage site, an X primer sequence and a promoter sequence;

(f) a step of preparing an amplified RNA (aRNA) complementary to the double-stranded cDNA sequence prepared in the step (e) with the double-stranded cDNA fragment as a template by means of an RNA polymerase recognizing the promoter sequence;

(g) a step of synthesizing a single-stranded cDNA having a sequence complementary to the X primer with the aRNA synthesized in the step (f) as a template;

(h) a step of synthesizing a double-stranded cDNA whose 5′ or 3′ end is fixed to solid phase or which has tag substance added to its 5′ or 3′ end with the single-stranded cDNA synthesized in the step (g) as a template;

(i) a step of cleaving the double-stranded cDNA synthesized in the step (h) with a second restriction enzyme Y that does not cleave the X primer sequence at its 5′ end;

(j) a step of purifying a double-stranded cDNA fragment comprising a cleavage site with the second restriction enzyme Y at its 3′ end from the fragments prepared in the step (i);

(k) a step of preparing a double-stranded cDNA fragment having a Y adaptor bound to its 3′ end by binding the X promoter-adaptor to a cleavage site with the second restriction enzyme Y in the double-stranded cDNA fragment purified in the step (j), wherein the Y adaptor comprises a sequence complementary to the cleavage site and a Y primer;

(l) a step of amplifying the double-stranded cDNA fragment prepared in the step (k) by means of PCR with the use of said double-stranded cDNA fragment as a template and a primer set of the X primer and Y primer;

(m) a step of PCR with the use of the double-stranded cDNA sequence prepared in the step (l) as a template and a primer set of X1 primer comprising two-base sequence of N₁N₂ (N₁ and N₂ are a base selected from the group consisting of adenine, thymine, guanine and cytosine, being the same or different with each other) at its 3′ end and Y1 primer comprising two base sequence of N₃N₄ (N₃ and N₄ are a base selected from the group consisting of adenine, thymine, guanine and cytosine, being the same or different with each other) at its 3′ end; and

(n) a step of subjecting the PCR product prepared in the step (m) to electrophoresis, and detecting migration length and peak so as to prepare the gene expression profile.

The above first method is characterized in that the amount of RNA comprised in a starting substance is increased by preparing an amplified RNA (aRNA) complementary to the double-stranded cDNA sequence by means of an RNA polymerase in the step (f), and that the number of the double-stranded cDNA having the X primer and Y primer added thereto is increased in the step (l).

The step (l) may be omitted in the case where a sufficient amount of aRNA for the analysis of gene expression profile can be obtained in the step (f) in the first method. A second method therefore is as follows:

A method for the preparation of gene expression profile comprising:

(a) a step of synthesizing a single-stranded cDNA whose 5′ end is fixed to solid phase or which has a tag substance added to its 5′ end with poly(A) RNA as a template;

(b) a step of synthesizing a double-stranded cDNA with the single-stranded cDNA synthesized in the step (a) as a template;

(c) a step of cleaving the double-stranded cDNA prepared in the step (b) with a first restriction enzyme X;

(d) a step of purifying a 3′ end fragment that is fixed to the solid phase or has the tag substance thereto from the double-stranded cDNA fragments prepared in the step (c);

(e) a step of preparing a double-stranded cDNA fragment having an X promoter-adaptor bound to its 5′ end by binding the X promoter-adaptor to a cleavage site with the first restriction enzyme X in the fragment purified in the step (d), wherein the X promoter-adaptor comprises a sequence complementary to the cleavage site, an X primer sequence and a promoter sequence;

(f) a step of preparing an amplified RNA (aRNA) complementary to the double-stranded cDNA sequence prepared in the step (e) with the double-stranded cDNA fragment as a template by means of an RNA polymerase recognizing the promoter sequence;

(g) a step of synthesizing a single-stranded cDNA having a sequence complementary to the X primer with the aRNA synthesized in the step (f) as a template;

(h) a step of synthesizing a double-stranded cDNA whose 5′ or 3′ end is fixed to solid phase or which has tag substance added to its 5′ or 3′ end with the single-stranded cDNA synthesized in the step (g) as a template;

(i) a step of cleaving the double-stranded cDNA synthesized in the step (h) with a second restriction enzyme Y that does not cleave the X primer sequence at its 5′ end;

(j) a step of purifying a double-stranded cDNA fragment comprising a cleavage site with the second restriction enzyme Y at its 3′ end from the fragments prepared in the step (i);

(k) a step of preparing a double-stranded cDNA fragment having a Y adaptor bound to its 3′ end by binding the X promoter-adaptor to a cleavage site with the second restriction enzyme Y in the double-stranded cDNA fragment purified in the step (j), wherein the Y adaptor comprises a sequence complementary to the cleavage site and a Y primer;

(m) a step of PCR with the use of the double-stranded cDNA sequence prepared in the step (l) as a template and a primer set of X1 primer comprising two-base sequence of N₁N₂ (N₁ and N₂ are a base selected from the group consisting of adenine, thymine, guanine and cytosine, being the same or different with each other) at its 3′ end and Y1 primer comprising two base sequence of N₃N₄ (N₃ and N₄ are a base selected from the group consisting of adenine, thymine, guanine and cytosine, being the same or different with each other) at its 3′ end; and

(n) a step of subjecting the PCR product prepared in the step (m) to electrophoresis, and detecting migration length and peak so as to prepare the gene expression profile.

The steps (f)-(h) may be further omitted in the case where a sufficient amount of the double-stranded cDNA for the analysis of gene expression profile can be amplified in PCR if the step (l) in the first method. A third method therefore is as follows:

A method for the preparation of gene expression profile comprising:

(a) a step of synthesizing a single-stranded cDNA whose 5′ end is fixed to solid phase, or which has tag substance added to its 5′ end with poly(A) RNA as a template;

(b) a step of synthesizing a double-stranded cDNA with the single-stranded cDNA synthesized in the step (a) as a template;

(c) a step of cleaving the double-stranded cDNA prepared in the step (b) with a first restriction enzyme X;

(d) a step of purifying a 3′ end fragment that is fixed to the solid phase or has the tag substance added thereto from the double-stranded cDNA fragments prepared in the step (c);

(e) a step of preparing a double-stranded cDNA fragment having an X promoter-adaptor bound to its 5′ end by binding the X promoter-adaptor to a cleavage site with the first restriction enzyme X in the fragment purified in the step (d), wherein the X promoter-adaptor comprises a sequence complementary to the cleavage site, an X primer sequence and a promoter sequence;

(i) a step of cleaving the double-stranded cDNA synthesized in the step (e) with a second restriction enzyme Y that does not cleave the X primer sequence at its 5′ end;

(j) a step of purifying a double-stranded cDNA fragment comprising a cleavage site with the second restriction enzyme Y at its 3′ end from the fragments prepared in the step (i);

(k) a step of preparing a double-stranded cDNA fragment having a Y adaptor bound to its 3′ end by binding the X promoter-adaptor to a cleavage site with the second restriction enzyme Y in the double-stranded cDNA fragment purified in the step (j), wherein the Y adaptor comprises a sequence complementary to the cleavage site and a Y primer;

(l) a step of amplifying the double-stranded cDNA fragment prepared in the step (k) by means of PCR with the use of said double-stranded cDNA fragment as a template and a primer set of the X primer and Y primer;

(m) a step of PCR with the use of the double-stranded cDNA sequence prepared in the step (l) as a template and a primer set of X1 primer comprising two-base sequence of N₁N₂ (N₁ and N₂ are a base selected from the group consisting of adenine, thymine, guanine and cytosine, being the same or different with each other) at its 3′ end and Y1 primer comprising two base sequence of N₃N₄ (N₃ and N₄ are a base selected from the group consisting of adenine, thymine, guanine and cytosine, being the same or different with each other) at its 3′ end; and

(n) a step of subjecting the PCR product prepared in the step (m) to electrophoresis, and detecting migration length and peak so as to prepare the gene expression profile.

It is preferable to carry out annealing of the X primer or X1 primer, and the Y primer or Y1 primer with the X adaptor and Y adaptor, respectively, in the step (l) and/or (m) at a temperature range of TmMAX+6° C.-TmMAX+14° C. of the primer, so that the occurrence of false peaks due to mis-annealing of the primers can be diminished. Unless otherwise noted in the specification, 5′ end of a sense chain (a chain homologous to a poly(A)RNA serving as a template) of a double-stranded cDNA means 5′ end of the double-stranded cDNA, and 3′ end of the sense chain means 3′ end of the double-stranded cDNA.

The “restriction enzyme” is an enzyme also called a “restriction endonuclease”, which will hydrolyze and cleave the double-stranded DNA at a particular sequence. Two kinds of the restriction enzymes “X” and “Y” are used in combination according to the present invention in order to obtain an appropriate fragment. It is preferable to use restriction enzymes in the present invention, which are able to cleave the double-stranded cDNA synthesized from mRNA of the expressed gene into fragments with an identifiable length. Further, it is preferable to use enzymes that can cleave as many double-stranded chains as possible, preferably almost all of them. For example, 6 or 4 base-recognizing enzymes known for those skilled in the art such as those described in WO02/48352 pamphlet may be used. As already described above, it is preferable to use the 4 base-recognizing enzymes such as MspI and MseI together in order to attain the high coverage ratio.

As the adaptor comprises a sequence complementary to the cleavage site of the first or second restriction enzyme, it can bind to the cleavage site. It also comprises the X primer sequence or Y primer sequence, so that a sequence located between these primers may be amplified in the step (l) by PCR using these primers. It may be optionally designed depending on the structure of the restriction enzymes and primers used in the reaction. The primers are usually 30-base long for performing a stable PCR.

The X primer, X1 primer, Y primer and Y1 primer have preferably 16 bases or more so as not to coincide with the subject RNA sequence wherever possible. Furthermore, it is necessary for these primers to satisfy the conditions generally required as a PCR primer, such as those described in “BioRad Experiment Illustrated (3) New Edition, Really Amplified PCR” Hiroki Nakayama, Shujunn Co., 2002, the second edition, the forth print. Each primer may be prepared in accordance with a general synthesizing method known for those skilled in the art (Letsinger et al., Nucleic Acids Research, 20, 1879-1882, 1992; Japanese Patent Application Publication Hei. 11(1999)-08018).

It is further preferable to bind a labeling substance such as any fluorescent substance known for those skilled in the art to at least either end of the primers in order to ease the detection after PCR. For example, the suitable fluorescent substances include 6-carboxyfluorescein (FAM), 4,7,2′,4′,5′,7′-hexachloro-6-carboxyfluorescein (HEX), NED (Applied System Japan, Co.) and 6-carboxy-X-rhodamine (Rox).

The degree of the amplification in the steps (f) and/or (l) of the present invention may be optionally determined by those skilled in the art depending on the starting substance, subject substance (the amount of mRNA originally comprised therein), the kind of polymerase and promoter sequence, and reaction conditions in each step. It is, however, necessary to maintain a ratio among the amounts of each mRNA originally comprised in the subject substance during the amplification steps(s) in order to accurately analyze the gene expression profile. For that purpose, it is preferable to obtain the amplified RNA in an amount of about 10 to 500 times as much as the number of the double-stranded cDNA fragments in the step (f). It is preferable to amplify the number of the double-stranded cDNA fragment by 128 to 1,024 times by repeating PCR in 7 to 10 cycles in the step (l).

There is no particular limitation on the RNA polymerase and promoter sequence used in the present invention and any ones known for those skilled in the art may be used. For example, there may be mentioned T3 or T7 promoter sequences derived from phage that infects E. coli, and SP6 promoter sequence, and RNA polymerases that can bind to these sequences.

When the double-stranded cDNA whose 5′ end is fixed to solid phase or which has tag substance added to its 5′ end is synthesized in the step (h) with the single-stranded cDNA synthesized in the step (g) as a template, an oligomer may be used as a primer for a complementarily synthesized cDNA, which comprises the X primer sequence or a part thereof fixed to the solid phase or having the tag substance added thereto. Alternatively, when the double-stranded cDNA whose 3′ end is fixed to solid phase or which has tag substance added to its 3′ end is synthesized in the step (h) with the single-stranded cDNA synthesized in the step (g) as a template, an oligo T primer fixed to the solid phase such as oligo T beads or having the tag substance added thereto may be used in the step (g) like in the step (a).

Furthermore, depending on the fact that the double-stranded cDNA fragment prepared in the step (h) is fixed via either 5′ end or 3′ end, or has the tag substance added to either its 5′ end or 3′ end, the double-stranded cDNA fragment fixed to the solid phase or having the tag substance shall be subjected to purification/collection or removal.

The solid phase may be optionally selected from any substances known for those skilled in the art, such as polystyrene beads, magnetic beads and silica-gel beads.

The tag substance and a substance having a high affinity for the tag substance mean one of the substances that can specifically bind with each other with a high affinity. Any substances may be used for them as long as they specifically bind with each other with a high affinity. Unlimited examples of the combination of these substances include biotin and streptavidin, biotin and avidin, FITC and anti-FITC antibody, DIG and anti-DIG, protein A and mouse IgG, and latex particles, etc. The tag substance may be added to the DNA sequence under any suitable conditions known for those skilled in the art. Each each sequence may be further fixed to the solid phase through the reaction between the tag substance and the substance having a high affinity for the tag substance.

The double-stranded cDNA fragment having the tag substance added thereto may be collected by means of a specific reaction between the tag substance and the substance having a high affinity for the tag substance. The double-stranded cDNA fragment fixed to the solid phase may be easily collected by removing other fragments from a reaction system with washing. These reactions may be carried out under any suitable conditions known for those skilled in the art.

Other conditions and apparatuses used in the HiCEP method may be referred to the description of WO02/48352 pamphlet. The resulting gene expression profile may be analyzed by means of any analyzing software known for those skilled in the art such as GeneScan (a trade mark: Applied BioSystems Japan, Co.)

A representative example of the base sequence (nucleotide sequence) of the transcript of the genes of Dusp4, Snk, Slc39a6 and Nnat is represented by SEQ ID No. 1, 2, 3 or 4, respectively.

Thus, the transcript of the gene may comprise the base sequence selected from the group consisting of:

(a) a base sequences represented by SEQ ID No. 1, 2, 3 and 4, respectively:

(b) a base sequence that hybridizes under strict conditions with a complementary base sequence of any one of the base sequence of (a); and

(c) a base sequence having homology of 80% or more with any one of the base sequence of (a).

The stringent conditions may be defined by an appropriate combination of the known factors such as salt concentration, organic solvent and temperature, being, for example, a temperature of 60-68° C., sodium concentration of 15-900 mM, preferably 15-600 mM and pH 6-8. One specific example of the stringent conditions includes hybridization under 5×SSC (750 mM NaCl, 75 mM tri-sodium citrate), 0.1% SDS, 5× Denhardt's solution, 50% formaldehyde at 42° C., followed by washing with 0.1×SSC (15 mM NaCl, 1.5 mM tri-sodium citrate), 0.1% SDS at 55° C.

Hybridization may be carried out by any method known for those skilled in the art such as those described in Current protocols in Molecular Biology (edited by Frederick M. Ausubel et al., 1987).

The base sequence that hybridizes under the strict conditions with a complementary base sequence of any one of the sequence represented by SEQ ID No. 1, 2, 3 or 4 may therefore have homology (identity) of 80% or more, preferably 90% or more, more preferably 95% or more, further preferably 99% or more with any one of the sequence represented by SEQ ID No. 1, 2, 3 or 4.

Alternatively, the detection of the expression level of the gene may be carried our in any manner known for those skilled in the art, for example, by determining an amount of one or more proteins encoded by the gene(s) or any part thereof (polypeptide) by means of any method known for those skilled in the art, such as, for example, an immuno-staining method such as a western blotting and EIA; an amino acid determination method using a gas-phase sequencer such as Edmon method; and mass spectrometry using MALDI-TOF/MS or ESI Q-TOF/MS.

Those skilled in the art may easily and optionally prepare an antibody used for the above methods by means of any conventional way using the above protein or polypeptide or a part thereof as an antigen. Polyclonal antibodies may be prepared by administering the antigen into a suitable animal such as mouse, rat, rabbit and goat and recovering antiserum form the animal. Monoclonal antibodies may be prepared in accordance with any known methods such as that described in “Monoclonal Antibody” James W. Goding, third edition, Academic Press, 1996.

The above protein may be therefore selected from the group consisting of:

(a) a protein encoded by the gene of Dusp4, Snk, Slc39a6 and Nnat, respectively;

(b) a protein having replacement or substitution, deletion or addition of one or a few amino acids in an amino acid sequence of any one of the protein of (a); and

(c) a protein consisting of an amino acid sequence having homology of 80% or more, preferably 90% or more, more preferably 95% or more, further preferably 99% or more with any one of the amino acid sequence of the protein of (a).

The homology or identity between the base or amino acid sequences may be determined by any method known for those skilled in the art, such as BLAST or FASTA program using Karlin and Altschul's algorithm (Proc. Natl. Acad. Sci., USA 87:2264-2268, 1990 and Proc. Natl. Acad. Sci., USA 90:5873-5877, 1993).

A kit used for the screening of the present invention may take any constitution or comprise any components in accordance with the determination method or mechanism used in the screening. The kit may include the primers or probes, antibodies, which may be optionally labeled with an appropriate labeling substance such as fluorescent substances, FAM (6-carboxyfluoroscein), HEX (4,7,2′,4′,5′,7′-hexachloro-6-carboxyfluorescein), NED (Applied Systems Japan) and Rox (6-carboxy-X-rhodamine), and other agents, enzymes, buffer solution, and reaction apparatus.

The substance that is screened by the above methods therefore includes a drug that is used for such a therapeutic purpose as to treat or prevent disorders or diseases related to or caused by any disorder or abnormality in the internal clock system, such as insomnia, disordered sleep, narcolepsy and jet syndrome.

### Examples

The present invention will be further illustrated more in detail with reference to the following examples, which shall not be construed to limit the scope of the present invention.

1. Materials and Methods

1.1. Preparation of Animals

Male C57BL/6 strain mice five weeks old purchased from SLC, Japan were kept in a cage (33 cm×21 cm) without running wheel for at least 10 days in a temperature controlled (23±2° C.) room under LD conditions (light for 12 hours, then dark for 12 hours), followed by three days and 14 hrs under DD conditions (constant darkness), then exposed to light continuously for 20 and 45 min and sacrificed with ether anesthesia. These preparation times were chosen because most known light-inducible transcripts, including the circadian rhythm-controlling genes, are induced by light exposure at those times [11]. In addition, we wanted to isolate the transcripts that are induced earliest after exposure in order to understand the first steps of the response to light exposure.

The time of light onset corresponds to circadian time (CT) 15, because the group mean of the free-running periods of the C57BL/6 mice colony in our experimental environment was 23.7 hrs (and therefore CT 15 in our mice closely corresponds to ZT 14 on day 4 of DD conditions). Control mice were killed at CT 15 without light exposure. Preliminary experiments with the Per1 gene have confirmed that the difference in mRNA abundance in the SCN of animals at CT 15 and at 45 min later is not statistically significant. To find whether induction occurs during subjective night or subjective day, we exposed the mice to light at CT 7, 15 and 23 and sacrificed them 45 min later. The light intensity was approximately 200 lux. To study circadian expression, on day 4 of DD conditions animals were sacrificed at CT 3, 7, 11, 15, 19, and 23. The SCN and cortex tissues were punched out from brain slices of approximately 1 mm in thickness using a puncher with an inner diameter of 400 micrometers. SCN tissues were obtained from the anterior section, and cortical tissues were obtained at 0.5 mm beneath the dorsal surface and 1 mm lateral to midline in the next caudal sections. Tissues from 10 (for each HiCEP reaction) or 5 (for real-time PCR) animals were pooled and immediately frozen in liquid nitrogen and stored at −80° C.

1.2. HiCEP Method

We performed HiCEP analysis as described in a previous report [3]. Three μg of total RNA treated with DNase I were used for each analysis. First-strand cDNA was synthesized with the SUPERSCRIPT™ II First-Strand Synthesis System for RT-PCR (Invitrogen) with 100 pmole of 5′ biotinylated oligo d(T) primer (5′-BTTTTTTTTTTTTTTTTTTV-3′) and the second strand was synthesized according to the protocol recommended by the manufacturer. The double strand cDNA (dscDNA) was digested with 50 units of MspI (TAKARA BIO, Ohtsu, Japan) followed by ligation of 5.0 μg of MspI-adapter with 400 units of T4 DNA Ligase (NEB, Beverly, Mass.). The ligated products with biotin at the 3′ terminus were collected by magnetic beads coated with Streptavidin (Dynabeads M-280 Streptavidin, DYNAL, Oslo, Norway) and washed twice with 1 ml of washing buffer (5 mM Tris-HCl pH 7.5, 0.5 mM EDTA, 1.0 M NaCl). The cDNA fragments were digested with 20 units of MseI (NEB) and the supernatants were collected. Ligation was performed with 10.2 pmole MseI-adapter using 400 units of T4 DNA Ligase in the presence of 2 units of MseI in 10 μl of reaction mixture. The resulting solution was used as a template for 256 selective PCR. In our selective PCR, the two nucleotides at either end of each target fragment, adjacent to the recognition sequences of the restriction enzymes, are annealed to the ends of PCR primers designed to segregate the fragment population into 256 subpopulations (two pairs of nucleotides, each with four possible nucleotides). The products were denatured and loaded on the electrophoresis of ABI PRISM 3100 (Applied Biosystems, Foster City, Calif., USA). Data produced by HiCEP were analyzed with the GeneScan 3.1.2 program (ABI).

The base sequences of the primers used in HiCEP are as follows:

| | |
|---|---|
| Dusp4: | |
| 5′-FAM-ACTCGGTTCATGACACGGAT-3′ | (SEQ ID NO: 5) |
| 5′-AGGCGTCCTACTGCGTAAAA-3′ | (SEQ ID NO: 6) |
| Snk: | |
| 5′-FAM-ACTCGGTTCATGACACGGAC-3′ | (SEQ ID NO: 7) |
| 5′-AGGCGTCCTACTGCGTAACC-3′ | (SEQ ID NO: 8) |
| Slc39a6: | |
| 5′-HEX-ACTCGGTTCATGACACGGCT-3′ | (SEQ ID NO: 9) |
| 5′-AGGCGTCCTACTGCGTAAGC-3′ | (SEQ ID NO: 10) |
| Nnat: | |
| 5′-NED-ACTCGGTTCATGACACGGGA-3′ | (SEQ ID NO: 11) |
| 5′-AGGCGTCCTACTGCGTAAGG-3′ | (SEQ ID NO: 12) |

### 1.3. Cloning of Peaks

The peaks of interest were fractionated with standard slab gel (20 cm×40 cm×4 mm). Collected fractions were re-amplified and cloned with a TA cloning kit (pGEM-T Easy, Promega, Madison, Wis.). To confirm that the cloned fragment is the fragment of interest, the length of the PCR products in the cloning vector was compared with that of the peak of interest in HiCEP using an ABI PRISM3100 electrophoresis. Sequencing was carried out by the dye terminator method.

1.4. Real-Time PCR

One microgram of total RNA prepared from the SCN and the cerebral cortex was treated with RNase-free DNase I (Invitrogen) and used for first strand cDNA synthesis (SUPERSCRIPT™ First-Strand Synthesis System for RT-PCR, Invitrogen). The reaction was performed according to the manufacturer's protocol using oligo-d(T) 18 primer. The 1st strand cDNA was diluted with 0.1× TE to 1/10 and used for the template of the quantitative PCR. The real-time PCR (SYBER GREEN PCR MASTER MIX, Applied Biosystems) was performed and analyzed by ABI PRISM 7700 (Applied Biosystems). The primers used in the real-time PCR for each gene are as shown in Table 2. The data were normalized in relation to the expression level of the glyceraldehyde phosphate dehydrogenase (Gapdh) gene. The PCR conditions were 95.0° C. for 10 min and 50 cycles of 95.0° C. for 15 sec, 60.0° C. for 30 sec and 78.0° C. for 40 sec.

1.5. In Situ Hybridization

Brains were fixed in 4% paraformaldehyde-PBS solution for 24 hrs, followed by immersion in 20% sucrose-PBS solution for another 24 hrs. Then the brains were sliced into 30-micrometer sections with a Cryostat (Leitz CM-1510, Germany). cDNA fragments were subcloned into the plasmid vectors, pGEM-T Easy (Promega) and linearized with restriction enzymes to be used as template for cRNA probes. Radio-labeled probes were prepared using [α-³³P]UTP (New England Nuclear, Boston, Mass., USA) with the standard protocols for cRNA synthesis. Hybridization was performed as described in a previous paper [12]. For the analyses, we used multiple probes for each gene as follows: for C/EBP beta, three fragments (−2-501, 513-1,022 and 822-1,357) of NM_{—}009883 (SEQ ID NO: 13); for Nr4a2, two fragments (715-1,219 and 1,295-1,794) of NM_{—}013613 (SEQ ID NO: 14); for Btg2, two fragments (505-1,041 and 1,077-1,601) of NM_007570 (SEQ ID NO:15); for Snk, two fragments (553-1,149 and 1,604-2,256) of NM_{—}152804.1 (SEQ ID NO:16). The first nucleotide “A” of the initial codon of open reading frame was defined as 1.

2. Results

2.1. Identification of Light-Inducible Genes From Mouse SCN

We investigated the changes of the transcriptome in response to light exposure in the SCN. Since the induction of known light-inducible genes begins within one hour after light exposure, we exposed mice to light at CT 15 and sacrificed 20 and 45 min later [12]. Three micrograms of total RNA purified from the SCN and treated with DNaseI were used for gene expression profiling analysis, and approximately 19,800 peaks were detected.

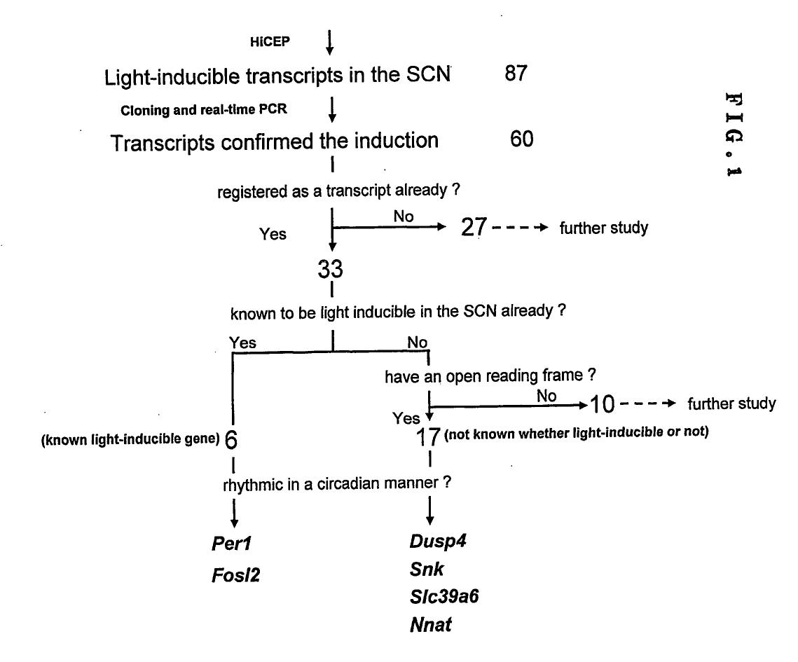

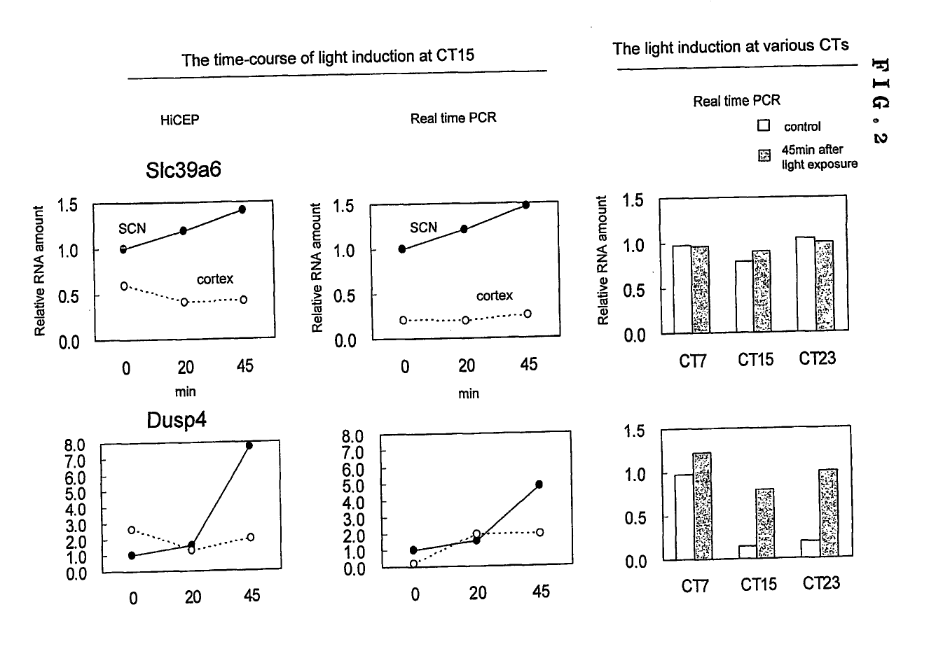

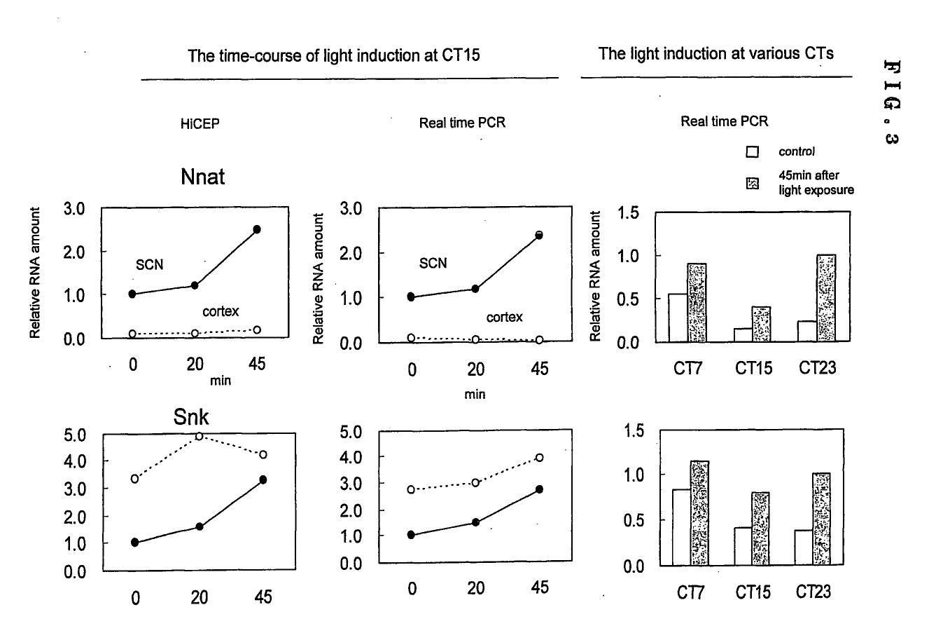

Results are shown in FIG. 1. We found 87 signal peaks that were induced (FIG. 2 and FIG. 3). We picked up the peaks whose change in gene expression depending on light exposure was more than 1.5 fold, because HiCEP discriminates 1.2-1.5 fold expression changes [3]. Subsequently, we tried to clone and sequence the light-induced peaks, followed by a BLAST search of the public nucleotide databases. To confirm the HiCEP results, we synthesized primers for each cloned transcript and performed real-time PCR with the RNA fractions, which were prepared independently from the samples for the HiCEP analyses. We confirmed that 60 of the transcripts are light-inducible (FIG. 2 and FIG. 3).

Thirty-three of the 60 confirmed transcripts were known and the remaining 27 were novel, and these are currently being investigated further. Only six of the 33 known transcripts, Nuclear Receptor Subfamily 4-group A-member 1 (Nr4a1), Per1, FBJ osteosarcoma oncogene B (FosB), Jun oncogene (Jun), FBJ osteosarcoma oncogene (c-Fos) and Fos-like antigen 2 (Fosl2), were known previously to be light-inducible in the mouse SCN (FIG. 1). In this invention, we focus on 17 known transcripts for which open reading frames have been suggested. We also found 10 transcripts assigned to intronic regions among the 33 known transcripts for which no open reading frames have been suggested. The information about the representative 4 light-inducible protein-coding transcripts that are newly identified here is summarized in Table 1 with the 2 known light-inducible transcripts as controls. All of the six known light-inducible genes encode transcription factors, and five transcription factor-encoding genes (including putative ones) were found in the 17 transcripts. Furthermore, phosphatase in the MAP kinase signal pathway and two types of kinase and ligase in the ubiquitin system were identified. No PAS domains were found in the products encoded by the genes. Domain-related information is shown in the InterPro column in Table 1.

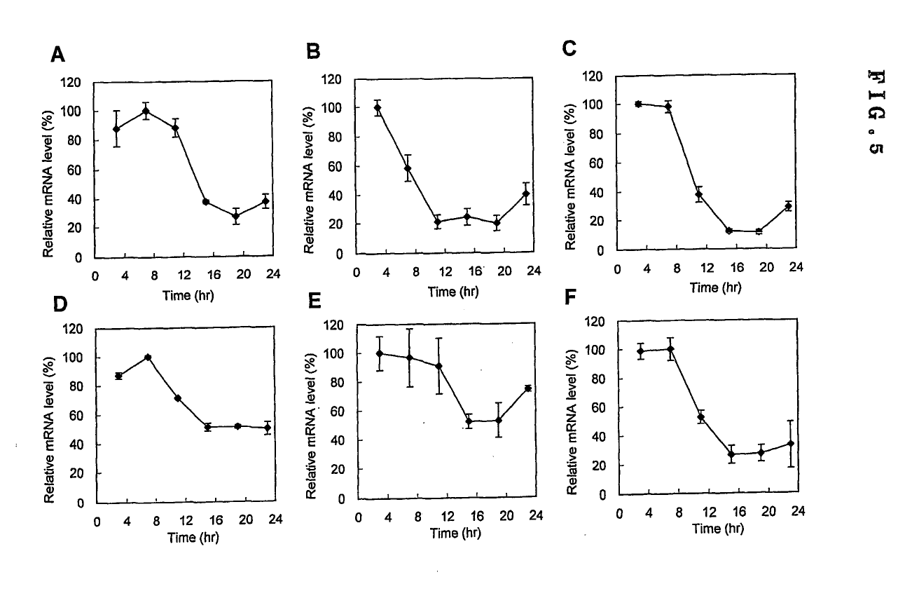

It is noteworthy that 11 of the 23 light-inducible genes in the SCN are also induced in the cortex. The genes exhibiting both light induction and rhythmic expression disclosed in our analysis show SCN-specific light induction (Table 1 and FIG. 5).

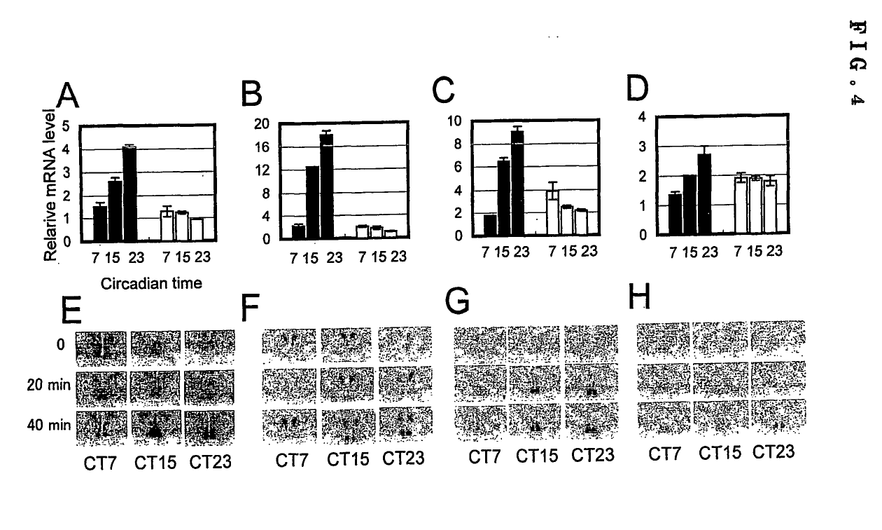

Next, we examined the 17 transcripts to learn whether induction occurs during subjective night or day. We found that 14 of them are light-inducible only during subjective night (FIG. 4A-D). In order to verify the HiCEP and real-time PCR results, and to investigate localization of the expression in the brain, we performed in situ hybridization with some randomly chosen transcripts and found localization in the SCN and subjective night-specific induction (FIG. 4E-H). Furthermore, we also detected significant Nr4a2 signals outside the SCN.

2.2. Light-Inducible and Rhythmically Expressed Transcripts

Per1 was among the light-inducible genes detected (Table 1). This is the key molecule for the core mechanism of circadian rhythm, and it is known to be a rhythmically expressed gene. We examined whether the 17 light-inducible genes and six control genes express rhythmically or not under conditions of constant darkness. We found that 6 known transcripts, Per1, Fosl2, Dusp4, Serum inducible kinase (Snk), solute carrier family 39-member 6 (Slc39a6, also called Liv1) and Neuronatin (Nnat) express rhythmically in the SCN (FIG. 5). All of these cycling genes reach peak expression during subjective day. This may be due to bias in the sample of transcripts examined: we chose transcripts that showed increased expression in response to light at CT 15, during subjective night.

The disclosure of the following references is incorporated herein by reference in its entirety.

### REFERENCES

[1] Albrecht, U., Sun, Z. S., Eichele, G. and Lee, C. C., A differential response of two putative mammalian circadian regulators, mper1 and mper2, to light, Cell, 91 (1997) 1055-1064.

[2] Evans, H. K., Wylie, A. A., Murphy, S. K. and Jirtle, R. L., The neuronatin gene resides in a “micro-imprinted” domain on human chromosome 20q 11.2, Genomics, 77 (2001) 99-104.

[3] Fukumura, R., Takahashi, H., Saito, T., Tsutsumi, Y., Fujimori, A., Sato, S., Tatsumi, K., Araki, R. and Abe, M., A sensitive transcriptome analysis method that can detect unknown transcripts, Nucleic Acids Res, 31 (2003) e94.

[4] Guido, M. E., Goguen, D., De Guido, L., Robertson, H. A. and Rusak, B., Circadian and photic regulation of immediate-early gene expression in the hamster suprachiasmatic nucleus, Neuroscience, 90 (1999) 555-571.

[5] Kagitani, F., Kuroiwa, Y., Wakana, S., Shiroishi, T., Miyoshi, N., Kobayashi, S., Nishida, M., Kohda, T., Kaneko-Ishino, T. and Ishino, F., Peg5/Neuronatin is an imprinted gene located on sub-distal chromosome 2 in the mouse, Nucleic Acids Res, 25 (1997) 3428-3432.

[6] Lee, H. S., Billings, H. J. and Lehman, M. N., The suprachiasmatic nucleus: a clock of multiple components, J Biol Rhythms, 18 (2003) 435-449.

[7] Nishimura, M., Yamagata, K., Sugiura, H. and Okamura, H., The activity-regulated cytoskeleton-associated (Arc) gene is a new light-inducible early gene in the mouse suprachiasmatic nucleus, Neuroscience, 116 (2003) 1141-1147.

[8] Obrietan, K., Impey, S. and Storm, D. R., Light and circadian rhythmicity regulate MAP kinase activation in the suprachiasmatic nuclei, Nat Neurosci, 1 (1998) 693-700.

[9] Pak, D. T. and Sheng, M., Targeted protein degradation and synapse remodeling by an inducible protein kinase, Science, 302 (2003) 1368-1373.

[10] Panda, S., Antoch, M. P., Miller, B. H., Su, A. I., Schook, A. B., Straume, M., Schultz, P. G., Kay, S. A., Takahashi, J. S. and Hogenesch, J. B., Coordinated transcription of key pathways in the mouse by the circadian clock, Cell, 109 (2002) 307-320.

[11] Schwartz, W. J., Carpino, A., Jr., de la Iglesia, H. O., Baler, R., Klein, D. C., Nakabeppu, Y. and Aronin, N., Differential regulation of fos family genes in the ventrolateral and dorsomedial subdivisions of the rat suprachiasmatic nucleus, Neuroscience, 98 (2000) 535-547.

[12] Shigeyoshi, Y., Taguchi, K., Yamamoto, S., Takekida, S., Yan, L., Tei, H., Moriya, T., Shibata, S., Loros, J. J., Dunlap, J. C. and Okamura, H., Light-induced resetting of a mammalian circadian clock is associated with rapid induction of the mPer1 transcript, Cell, 91 (1997) 1043-1053.

[13] Simmons, D. L., Neel, B. G., Stevens, R., Evett, G. and Erikson, R. L., Identification of an early-growth-response gene encoding a novel putative protein kinase, Mol Cell Biol, 12 (1992) 4164-4169.

[14] Takahashi, H., Umeda, N., Tsutsumi, Y., Fukumura, R., Ohkaze, H., Sujino, M., van der Horst, G., Yasui, A., Inouye, S. T., Fujimori, A., Ohhata, T., Araki, R. and Abe, M., Mouse dexamethasone-induced RAS protein 1 gene is expressed in a circadian rhythmic manner in the suprachiasmatic nucleus, Brain Res Mol Brain Res, 110 (2003) 1-6.

[15] Taylor, K. M. and Nicholson, R. I., The LZT proteins; the LIV-1 subfamily of zinc transporters, Biochim Biophys Acta, 1611(2003)16-30.

### INDUSTRIAL APPLICABILITY

The present methods provide a substance that is advantageously used for such a therapeutic purpose as to treat or prevent disorders or diseases related to or caused by any disorder or abnormality in the internal clock system, such as insomnia, disordered sleep, narcolepsy and jet syndrome.

1. A method for screening a substance that affects an internal clock system in an organism, comprising the detection of the expression level of a gene exhibiting a light-inducible and rhythmic expression in a circadian manner.

2. The method for screening according to claim 1, based on change in the expression level of the gene exhibiting a light-inducible and rhythmic expression in a circadian manner in the presence of the substance.

3. The method for screening according to claim 1, the gene is expressed in the suprachiasmatic nucleus of mammalian.

4. The method of screening according to claim 1, the gene is selected from the group consisting of Dusp4, Snk, Slc39a6, Nnat and any combination thereof.

5. The method of screening according to claim 1, comprising the steps of:
(a) contacting a cultured cell line derived from the suprachiasmatic nucleus in a brain with a substance to be screened;
(b) detecting change of the expression level of the gene; and
(c) selecting the substance that affects the expression level of the gene.

6. The method of screening according to claim 1, wherein the expression level of a gene is detected by determining an amount of a transcript of the gene.

7. The method of screening according to claim 6, wherein the amount of the transcript of the gene is determined by obtaining a gene expression profile by means of HiCEP procedure.

8. The method of screening according to claim 6, wherein the amount of the transcript of the gene is determined by PCR.

9. The method according to claim 7, wherein the transcript of the gene comprises the base sequence selected from the group consisting of:
(a) a base sequences represented by SEQ ID No. 1, 2, 3 and 4, respectively:
(b) a base sequence that hybridizes under strict conditions with a complementary base sequence of any one of the base sequence of (a); and
(c) a base sequence consisting of homology of 80% or more with any one of the base sequence of (a).

10. The method of screening according to claim 1, wherein the expression level of a gene is detected by determining an amount of one or more proteins encoded by the gene or any part thereof.

11. The method for screening according to claim 5, the protein is selected from the group consisting of:
(a) a protein encoded by the gene of Dusp4, Snk, Slc39a6 and Nnat, respectively;
(b) a protein having replacement or substitution, deletion or addition of one or a few amino acids in an amino acid sequence of any one of the protein of (a); and
(c) a protein consisting of an amino acid sequence having homology of 80% or more with any one of the amino acid sequence of the protein of (a).

12. The method of screening according to claim 2, wherein the expression level of a gene is detected by determining an amount of a transcript of the gene.

13. The method of screening according to claim 3, wherein the expression level of a gene is detected by determining an amount of a transcript of the gene.

14. The method of screening according to claim 4, wherein the expression level of a gene is detected by determining an amount of a transcript of the gene.

15. The method of screening according to claim 5, wherein the expression level of a gene is detected by determining an amount of a transcript of the gene.

16. The method according to claim 8, wherein the transcript of the gene comprises the base sequence selected from the group consisting of:
(a) a base sequences represented by SEQ ID No. 1, 2, 3 and 4, respectively:
(b) a base sequence that hybridizes under strict conditions with a complementary base sequence of any one of the base sequence of (a); and
(c) a base sequence consisting of homology of 80% or more with any one of the base sequence of (a).

17. The method of screening according to claim 2, wherein the expression level of a gene is detected by determining an amount of one or more proteins encoded by the gene or any part thereof.

18. The method of screening according to claim 3, wherein the expression level of a gene is detected by determining an amount of one or more proteins encoded by the gene or any part thereof.

19. The method of screening according to claim 4, wherein the expression level of a gene is detected by determining an amount of one or more proteins encoded by the gene or any part thereof.

20. The method of screening according to claim 5, wherein the expression level of a gene is detected by determining an amount of one or more proteins encoded by the gene or any part thereof.


SEQUENCE LISTING (12 sequences)

SEQ ID NO: 1 - DNA, 80 bp, Mus musculus
ccggatgccagctcctgttttactgatatagggtgacttaggagccatgctgggttccaactgggccaacaccattttaa

SEQ ID NO: 2 - DNA, 205 bp, Mus musculus
ccggacaaaaagacagttcactattatgcggaacttggccaatgctctgttttcccagcaacagatgcccctgaacaatttattagtcaagtgacggtgctgaaatacttttctcattacatggaggagaacctcatggatggtggtgatctcccgagtgttactgacattcgaagacctcggctctacctcctgcagtggttaa

SEQ ID NO: 3 - DNA, 216 bp, Mus musculus
ccggcttgttcatgtatgtcgctctggttgacatggtgcctgagatgttgcacaatgatgctagtgatcacggatgcagccgttggggatatttcttcctgcagaatgctgggatacttctcggttttggaattatgttactcatttccatatttgagcataaaattgtgtttcgtataaatttctaattaggttctaaaccctagagcagcttaa

SEQ ID NO: 4 - DNA, 163 bp, Mus musculus
ccgggaacaaagactcaggggtgcggcggtcgcccgctgcggacgatcgcgaccaacgcggtaagaaatacccgctatattcaggctcgatcccaggagggaggcggggcacttgtgtgctgtgaaactttacagttcgcacaacagaaaattttcaccttaa

SEQ ID NO: 5 - DNA, 20 bp, Artificial Sequence
  primer
actcggttcatgacacggat

SEQ ID NO: 6 - DNA, 20 bp, Artificial Sequence
  primer
aggcgtcctactgcgtaaaa

SEQ ID NO: 7 - DNA, 20 bp, Artificial Sequence
  primer
actcggttcatgacacggac

SEQ ID NO: 8 - DNA, 20 bp, Artificial Sequence
  primer
aggcgtcctactgcgtaacc

SEQ ID NO: 9 - DNA, 20 bp, Artificial Sequence
  primer
actcggttcatgacacggct

SEQ ID NO: 10 - DNA, 20 bp, Artificial Sequence
  primer
aggcgtcctactgcgtaagc

SEQ ID NO: 11 - DNA, 20 bp, Artificial Sequence
  primer
actcggttcatgacacggga

SEQ ID NO: 12 - DNA, 20 bp, Artificial Sequence
  primer
aggcgtcctactgcgtaagg